(19) United States
(12) Patent Application Publication
(10) Pub. No.: US 2009/0171186 A1
(43) Pub. Date: Jul. 2, 2009

(54) MRI APPARATUS AND MAGNETIC RESONANCE IMAGING METHOD

(76) Inventor: Naoyuki Takei, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/343,072

(22) Filed: Dec. 23, 2008

(30) Foreign Application Priority Data
Dec. 28, 2007 (JP) 2007-340864

(51) Int. Cl. A61B 5/055

(52) U.S. Cl. 600/419

(57) ABSTRACT

An MRI apparatus acquires a blood signal about blood from a subject having the blood flowing from a first region to a second region via an imaging region. The apparatus includes a first longitudinal magnetization inverting device for inverting a longitudinal magnetization direction of the blood in the first region during a first inversion period, a second longitudinal magnetization inverting device that inverts a longitudinal magnetization direction of the blood in process of longitudinal magnetization recovery and inverts a longitudinal magnetization direction of tissues in the imaging region and the second region during a second inversion period after the first inversion period, and a data acquisition device that acquires a blood signal about blood flowing from the first region to the imaging region during a data acquisition period after the second inversion period.

### CROSS REFERENCE TO RELATED APPLICATIONS

This application claims the benefit of Japanese Patent Application No. 2007-340864 filed Dec. 28, 2007, which is hereby incorporated by reference in its entirety.

### BACKGROUND OF THE INVENTION

The embodiments described herein relate to an MRI apparatus for imaging a blood flow and a magnetic resonance imaging method for a blood flow.

Conventionally, an MRI apparatus is used to image a blood flow in a blood vessel. For example, the Time-SLIP technique is known as a method of imaging a blood flow (see Image Information Medical, September 2006, pp. 952-957).

The method described above may narrow a rendered blood flow range when imaging a patient whose blood flow is slow.

It is desirable that the problem described previously is solved.

### BRIEF DESCRIPTION OF THE INVENTION

An MRI apparatus acquires a blood signal about blood from a subject having the blood flowing from a first region to a second region via an imaging region and includes: a first longitudinal magnetization inverting device for inverting a longitudinal magnetization direction of the blood in the first region during a first inversion period; a second longitudinal magnetization inverting device that inverts a longitudinal magnetization direction of the blood in process of longitudinal magnetization recovery and inverts a longitudinal magnetization direction of tissues in the imaging region and the second region during a second inversion period after the first inversion period; and a data acquisition device that acquires a blood signal about blood flowing from the first region to the imaging region during a data acquisition period after the second inversion period.

During the second inversion period, the second longitudinal magnetization inverting device inverts a longitudinal magnetization direction of the blood in process of longitudinal magnetization recovery and inverts a longitudinal magnetization direction of a tissue in the imaging region and the second region. At the end of the second inversion period, the longitudinal magnetization component Mz of the imaging region and the second region is set to −1. The longitudinal magnetization component Mz of blood in process of longitudinal magnetization recovery is in the range of −1<Mz<1. Accordingly, the blood can be more intensely rendered than the other tissues by starting a data acquisition period at a time point when the longitudinal magnetization for the tissue in the imaging region and the second region reaches or approximately reaches a null point.

Embodiments of the invention invert the longitudinal magnetization direction of a blood flow before inverting the longitudinal magnetization direction of the tissue in the imaging region and the second region. Accordingly, the time for inverting the longitudinal magnetization direction of blood until starting the data acquisition (a total of the first and second inversion times) becomes longer than the time period from a time after inverting the longitudinal magnetization direction of the tissue in the imaging region and the second region to a time of stating the data acquisition (the second inversion time). Accordingly, the blood can be rendered in a wide range even at a low blood flow rate.

Further objects and advantages of the present invention will be apparent from the following description of the embodiments of the invention as illustrated in the accompanying drawings.

### BRIEF DESCRIPTION OF THE DRAWINGS

FIG. 1 is an example of a block diagram showing the MRI apparatus 100;

FIG. 2 is an example of a function block diagram of the computer 107;

FIG. 3 shows a process flow of the MRI apparatus 100;

FIG. 4 schematically shows the imaging region FOV of the subject 10;

FIG. 5 shows an example of the pulse sequence performed at Step S12;

FIG. 6A is a graph showing the longitudinal magnetization component Mz of the arterial blood AR of the subject at time t1 of the pulse sequence 60 in FIG. 5, and FIG. 6B is a graph showing the longitudinal magnetization component Mz of the venous blood VE of the subject 10 at time t1 of the pulse sequence 60 in FIG. 5;

FIG. 7A is a graph showing the longitudinal magnetization component Mz of the arterial blood AR of the subject at time t2 of the_pulse sequence 60 in FIG. 5, and FIG. 7B is a graph showing the longitudinal magnetization component Mz of the venous blood VE of the subject 10 at time t2 of the pulse sequence 60 in FIG. 5;

FIG. 8A is a graph showing the longitudinal magnetization component Mz of the arterial blood AR of the subject at time t3 of the pulse sequence 60 in FIG. 5, and FIG. 8B is a graph showing the longitudinal magnetization component Mz of the venous blood VE of the subject 10 at time t3 of the pulse sequence 60 in FIG. 5;

FIG. 9A is a graph showing the longitudinal magnetization component Mz of the arterial blood AR of the subject at time t4 of the pulse sequence 60 in FIG. 5, and FIG. 9B is a graph showing the longitudinal magnetization component Mz of the venous blood VE of the subject 10 at time t4 of the pulse sequence 60 in FIG. 5;

FIG. 10A is a graph showing the longitudinal magnetization component Mz of the arterial blood AR of the subject at time t5 of the pulse sequence 50 in FIG. 5, and FIG. 10B is a graph showing the longitudinal magnetization component Mz of the venous blood VE of the subject 10 at time t5 of the pulse sequence 50 in FIG. 5;

FIGS. 11A and 11B show pulse sequences according to the embodiment and the Time-SLIP technique;

FIGS. 12A and 12B are graphs showing the longitudinal magnetization component Mz of the arterial blood AR of the subject 10 at time t1 of the pulse sequences 50 and 51 in FIG. 11;

FIGS. 13A and 13B are graphs showing the longitudinal magnetization component Mz of the arterial blood AR of the subject 10 at time t2 of the pulse sequences 50 and 51 in FIG. 11;

FIGS. 14A and 14B are graphs showing the longitudinal magnetization component Mz of the arterial blood AR of the subject 10 at time t3 of the pulse sequences 50 and 51 in FIG. 11;

FIGS. 15A and 15B are graphs showing the longitudinal magnetization component Mz of the arterial blood AR of the subject 10 at time t4 of the pulse sequences 50 and 51 in FIG. 11;

FIGS. 16A and 16B are graphs showing the longitudinal magnetization component Mz of the arterial blood AR of the subject 10 at time t4′ of the pulse sequences 50 and 51 in FIG. 11;

FIGS. 17A and 17B are graphs showing the longitudinal magnetization component Mz of the arterial blood AR of the subject 10 at time t5 of the pulse sequences 50 and 51 in FIG. 11;

FIG. 18 shows the longitudinal magnetization component Mz of the arterial blood AR at time t3 when the inversion times TIa are set to 840 ms and 600 ms;

FIG. 19 shows the longitudinal magnetization component Mz of the arterial blood AR immediately after the second inversion period IR2 (time t4 in FIG. 5); and

FIG. 20 shows the longitudinal magnetization component Mz of the arterial blood AR at the start time of the data acquisition period ACQ (time 5 in FIG. 5).

### DETAILED DESCRIPTION OF THE INVENTION

Embodiments of the present invention will be described in further detail with reference to the accompanying drawings. The invention is not limited to the embodiments described herein.

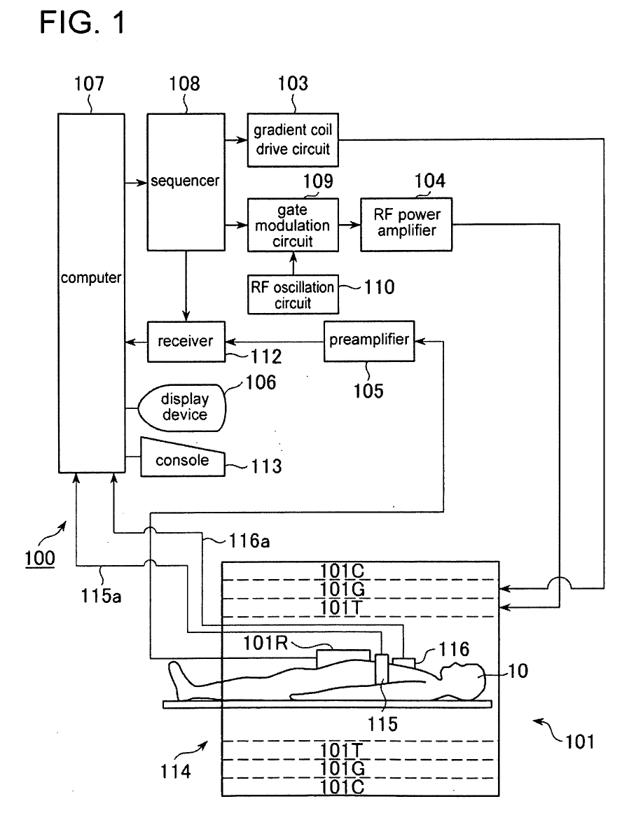

FIG. 1 is an example of a block diagram showing an exemplary MRI (Magnetic Resonance Imaging) apparatus 100.

The MRI apparatus 100 includes a magnet assembly 101. The magnet assembly 101 has a bore 114 for inserting a subject 10. The magnet assembly 101 also includes a static magnetic field coil 101C, a gradient coil 101G, and a transmission coil 101T.

The static magnetic field coil 101C forms a constant static magnetic field to the inside of the bore 1 14. The gradient coil 101G is connected to a gradient coil drive circuit 103 and generates gradient magnetic fields along X, Y, and Z axes. The transmission coil 101T is connected to an RF power amplifier 104 and supplies an RF pulse to the inside of the bore 114.

The MRI apparatus 100 includes a bellows 115 and a heartbeat sensor 116.

The bellows 115 detects an aspiration of the subject 10 and transmits an aspiration signal 115a to a computer 107. The heartbeat sensor 116 detects a heartbeat of the subject 10 and transmits an electrocardiographic signal 116a to the computer 107.

The computer computes aspiration and heartbeat states of the subject 10 based on the received aspiration signal 115a and electrocardiographic signal 116a and outputs a computation result to a sequencer 108.

The sequencer 108 controls the gradient coil drive circuit 103 and a gate modulation circuit 109 in accordance with an instruction received from the computer 107. The gradient coil drive circuit 103 drives a gradient coil 101G in accordance with an instruction from the sequencer 108. As a result, the gradient coil 101G applies a gradient pulse to the subject 10. The gate modulation circuit 109 modulates a carrier wave from an RF oscillation circuit 110 in accordance with an instruction from the sequencer 108 and outputs the modulation signal to the RF power amplifier 104. The RF power amplifier 104 amplifies the modulation signal and supplies the modulation signal to the transmission coil 101T. As a result, the transmission coil 101T applies a transmission pulse to the subject 10.

The MRI apparatus 100 includes a reception coil 101R. The reception coil 101R is connected to a preamplifier 105 and receives an MR signal from the subject 10. The MR signal is amplified by the preamplifier 105 and is supplied to a receiver 112. The receiver 112 converts the amplified MR signal into a digital data and outputs the digital data to the computer 107.

The computer 107 reconstructs an image based on the digital data from the receiver 112. The reconstructed image is displayed on a display device 106. An operator of the MRI apparatus 100 can interactively operate the MRI apparatus 100 using the display device 106 and a console 113.

The following describes operation of the MRI apparatus 100 constituted as mentioned above.

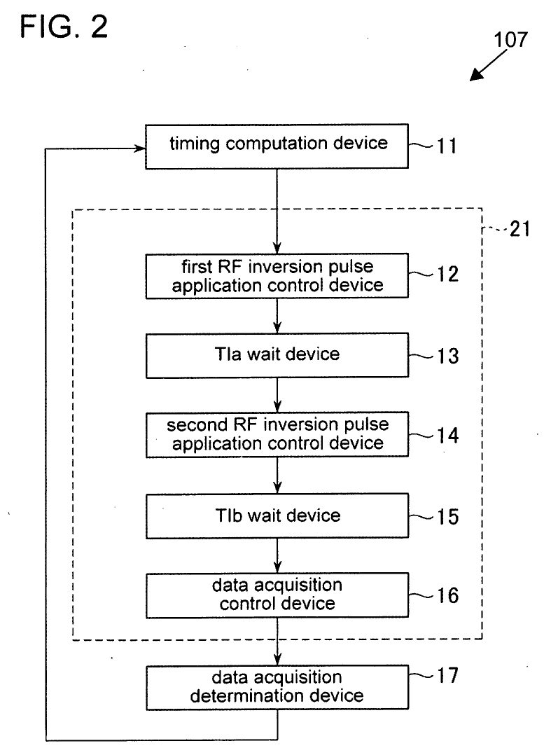

FIG. 2 is an example of a function block diagram of the computer 107.

The computer 107 includes a timing computation device 11, a pulse sequence execution control device 21, and a data acquisition determination device 17.

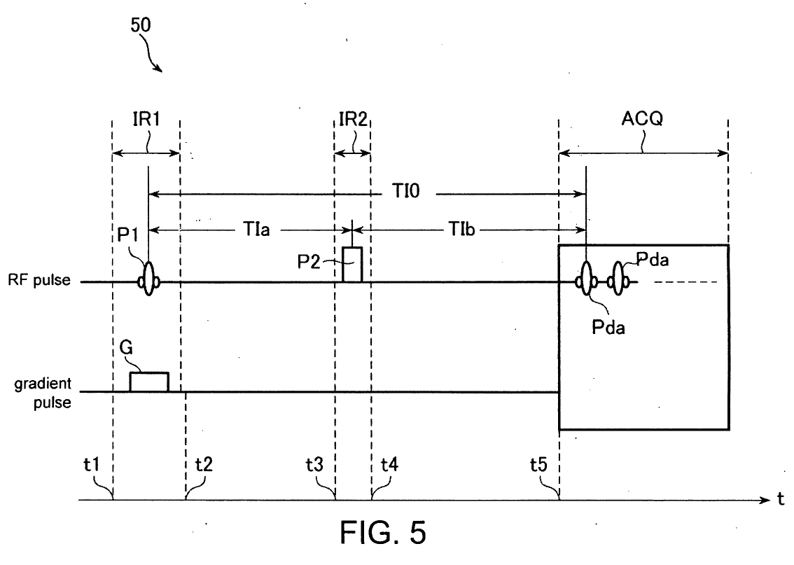

The timing computation device 11 computes a timing to start a pulse sequence (see FIG. 5 to be described later) based on the aspiration signal 115a and the electrocardiographic signal 116a (see FIG. 1). The pulse sequence execution control device 21 controls execution of a pulse sequence. The data acquisition determination device 17 determines whether or not to continue data acquisition.

For controlling pulses sequent execution, the pulse sequence execution control device 21 includes five function blocks (a first RF inversion pulse application control device 12, a TIa wait device 13, a second RF inversion pulse application control device 14, a TIb wait device 15, and a data acquisition control device 16).

The first RF inversion pulse application control device 12 supplies the sequencer 108 with an instruction for applying a first inversion pulse P1 (see FIG. 5 to be described later) to the subject 10. The second RF inversion pulse application control device 14 supplies the sequencer 108 with an instruction for applying a second inversion pulse P2 (see FIG. 5 to be described later) to the subject 10. The data acquisition control device 16 supplies the sequencer 108 with an instruction for collecting an MR signal from the subject 10. The pulse sequence execution control device 21 has two wait devices 13 and 15. TIa wait device 13 supplies the sequencer 108 with an instruction for ensuring a wait time (first reverse time TIa) between the first inversion pulse P1 and the second inversion pulse P2. TIb wait device 15 supplies the sequencer 108 with an instruction for ensuring a wait time (second reverse time TIb) between the second inversion pulse P2 and an excitation pulse Pda (see FIG. 5 to be described later) for a data acquisition period.

The following describes processes performed by the MRI apparatus 100 in a case of imaging arterial blood of the subject 10.

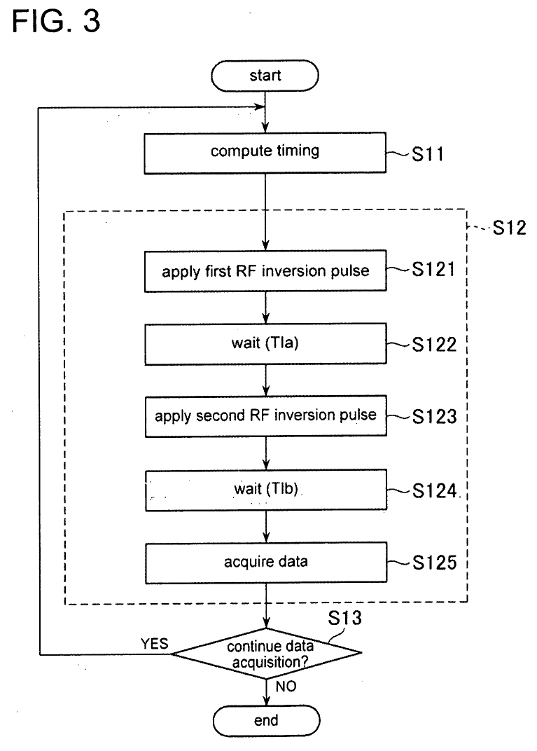

FIG. 3 shows a process flow of the MRI apparatus 100.

At Step S11, the process computes a timing to start a pulse sequence (see FIG. 5 to be described later) based on the aspiration signal 115a and the electrocardiographic signal 116a (see FIG. 1). After Step S11, the process proceeds to Step S12.

At Step S12, the process performs the pulse sequence (see FIG. 5 to be described later) to collect data about arterial blood AR from an imaging region FOV of the subject 10.

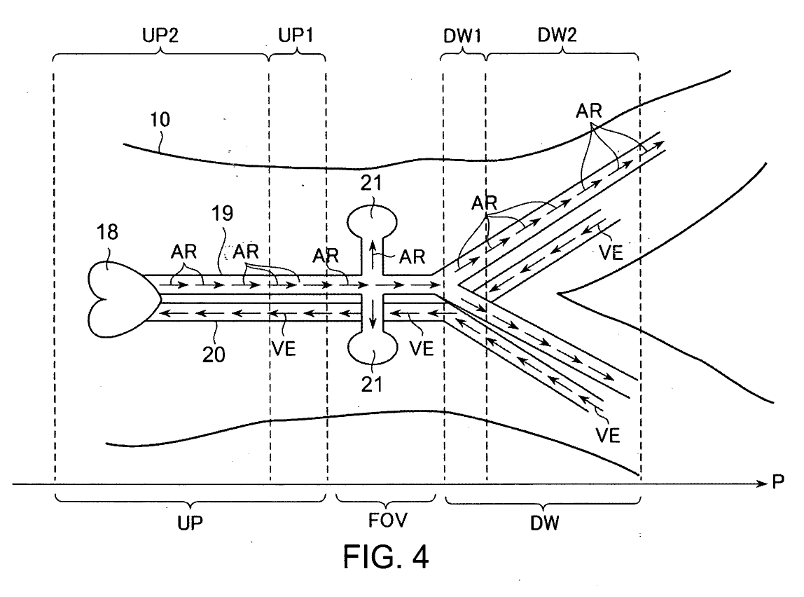

FIG. 4 schematically shows the imaging region FOV of the subject 10.

FIG. 4 shows an artery 19 and a vein 20 connecting with a heart 18 of the subject 10. The artery 19 also connects with a kidney 21. According to the embodiment, the imaging region FOV includes the kidney 14. The heart 18 supplies the arterial blood to the artery 19. The arterial blood AR flows from an upstream region UP to a downstream region DW via the imaging region FOV. The venous blood VE flows from the downstream region DW to the upstream region UP via the imaging region FOV contrary to the arterial blood AR.

At Step S12, the process collects the data about the arterial blood AR and then proceeds to Step S13 and determines whether or not to further collect data. To continue collecting data, the process returns to Step S11. The loop terminates when it is determined not to continue collecting data at Step S13.

The venous blood VE as well as the arterial blood AR flows through the imaging region FOV. The imaging region FOV further contains motionless tissues (e.g., a muscle and a kidney 21). The embodiment aims at rendering the arterial blood AR. It is difficult to visually check a blood flow state of the arterial blood AR when the venous blood VE and the kidney 21 are rendered along with the arterial blood AR. There is need to possibly avoid rendering tissues (such as the venous blood VE and the kidney 21) not targeted for imaging. The embodiment performs the following pulse sequence at Step S12 to possibly avoid rendering tissues (such as the venous blood VE and the kidney 21) not targeted for imaging.

FIG. 5 shows an example of the pulse sequence performed at Step S12.

The pulse sequence 50 includes a first inversion period IR1, a second inversion period IR2, and a data acquisition period ACQ.

During the first inversion period IR1, the gradient coil 101G (see FIG. 1) applies a gradient pulse G to the subject 10. While the gradient pulse G is applied, the transmission coil 101T applies a selective RF inversion pulse P1. The gradient pulse G and the selective RF inversion pulse P1 are so designed as to invert a longitudinal magnetization direction of a tissue in the upstream region UP (see FIG. 4). The first inversion period IR1 is followed by the second inversion period IR2.

During the second inversion period IR2, the transmission coil 101T applies a nonselective RF pulse P2 to the subject 10. The nonselective RF pulse P2 is applied at the time point where the first inversion time TIa has elapsed after application of a selective RF pulse P1. The second inversion period IR2 is followed by the data acquisition period ACQ.

Data is acquired during the data acquisition period ACQ. During the data acquisition period ACQ, the transmission coil 101T applies many excitation pulses Pda. The transmission coil 101T starts applying an excitation pulse Pda at the time point where a second inversion time TIb has elapsed after transmission of a nonselective RF inversion pulse P2.

Performing the pulse sequence 50 in FIG. 5 makes it possible to obtain a blood flow image with the arterial blood AR emphasized. To perform the pulse sequence in FIG. 5, Step S12 includes Sub-steps S121 through S125 as shown in FIG. 3.

At Sub-step S121, the process applies the first inversion pulse P1 to the subject 10 during the first inversion period IR1. At Sub-step S123, the process applies the second inversion pulse P2 to the subject 10 during the second inversion period IR. At Sub-step S125, the process acquires an MR signal from the subject 10 during the data acquisition period ACQ. Sub-step S122 is provided between the Sub-step S121 and S123. Sub-step S124 is provided between the Sub-step S123 and S125. At Sub-step S122, the first inversion time TIa is ensured as a wait time between the first inversion pulse P1 and the second inversion pulse P2. At Sub-step S124, the second inversion time TIb is ensured as a wait time between the second inversion pulse P2 and the excitation pulse Pda.

By performing the pulse sequence 50 in FIG. 5, the MRI apparatus 100 can obtain a blood flow image with the arterial blood AR emphasized. With reference to FIGS. 5 and 6 through 10, the following describes why performing the pulse sequence 50 makes it possible to obtain a blood flow image with the arterial blood AR emphasized.

FIGS. 6 through 10 are graphs showing longitudinal magnetization components Mz of the arterial blood AR and the venous blood VE of the subject 10 at time points of the pulse sequence 50 in FIG. 5.

Horizontal axes of the graphs in FIGS. 6 through 10 indicate positions P in the upstream region UP, the imaging region FOV, and the downstream region DW (see FIG. 4). The horizontal axis (position P) of the graph indicates the entire imaging region FOV. With respect to the upstream region UP, the horizontal axis indicates only the first upstream region UP1 (see FIG. 4) approximate to the imaging region FOV. With respect to the downstream region DW, the horizontal axis indicates only the first downstream region DW1 (see FIG. 4) approximate to the imaging region FOV. Vertical axes of the graphs in FIGS. 6 through 10 indicate longitudinal magnetization components Mz of the arterial blood AR and the venous blood VE of the subject 10.

FIGS. 6A and 6B indicate longitudinal magnetization components Mz of the arterial blood AR and the venous blood VE immediately before the first inversion period IR1 (time t1 in FIG. 5). The graph in FIG. 6A shows a line A1 that represents relation between the position P and the longitudinal magnetization component Mz of the arterial blood AR at time t1. The graph in FIG. 6B shows a line V1 that represents relation between the position P and the longitudinal magnetization component Mz of the venous blood VE at time t1.

At time t1, the first inversion period IR1 does not start. Accordingly, the longitudinal magnetization component Mz of the arterial blood AR is set to 1 throughout the upstream region UP (first and second upstream regions UP1 and UP2), the imaging region FOV, and the downstream region DW (first and second downstream regions DW1 and DW2).

The longitudinal magnetization component Mz of the venous blood VE is also set to 1 throughout the upstream region UP (first and second upstream regions UP1 and UP2), the imaging region FOV, and the downstream region DW (first and second downstream regions DW1 and DW2).

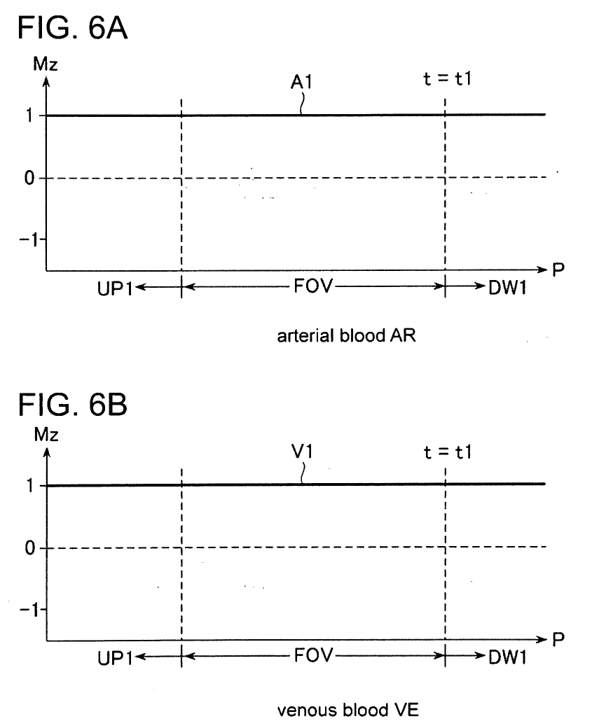

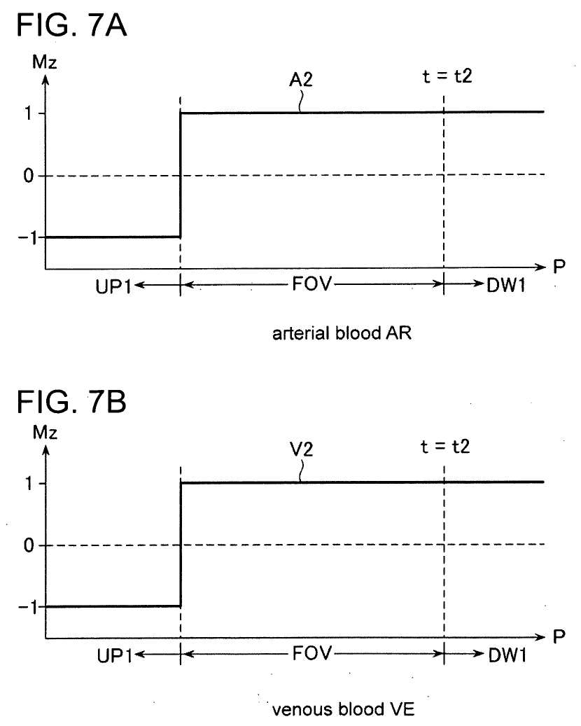

The first inversion period IR1 starts immediately after time t1 (see FIG. 5). The gradient pulse G and the selective RF inversion pulse P1 are applied during the first inversion period IR1. The gradient pulse G and the selective RF inversion pulse P1 are so designed as to invert a longitudinal magnetization direction of a tissue in the upstream region UP (see FIG. 4). Accordingly, the longitudinal magnetization direction of the tissue in the upstream region UP is inverted during the first inversion period IR1. As a result, the longitudinal magnetization components Mz of the arterial blood AR and the venous blood VE change as shown in graphs of FIG. 7 immediately after the first inversion period IR1 (time t2).

FIGS. 7A and 7B show the longitudinal magnetization components Mz of the arterial blood AR and the venous blood VE immediately after expiration of the first inversion period IR1 (time t2 in FIG. 5). The graph in FIG. 7A shows a line A2 that represents relation between the position P and the longitudinal magnetization component Mz of the arterial blood AR at time t2. The graph in FIG. 7B shows a line V2 that represents relation between the position P and the longitudinal magnetization component Mz of the venous blood VE at time t2.

The longitudinal magnetization direction of the tissue in the upstream region UP is inverted during the first inversion period IR1. The longitudinal magnetization component Mz of the arterial blood AR is inverted to −1 from 1 in the upstream region UP (first and second upstream regions UP1 and UP2).

Similarly to the longitudinal magnetization component Mz of the arterial blood AR, the longitudinal magnetization component Mz of the venous blood VE is also inverted to −1 from 1 in the upstream region UP (first and second upstream regions UP1 and UP2).

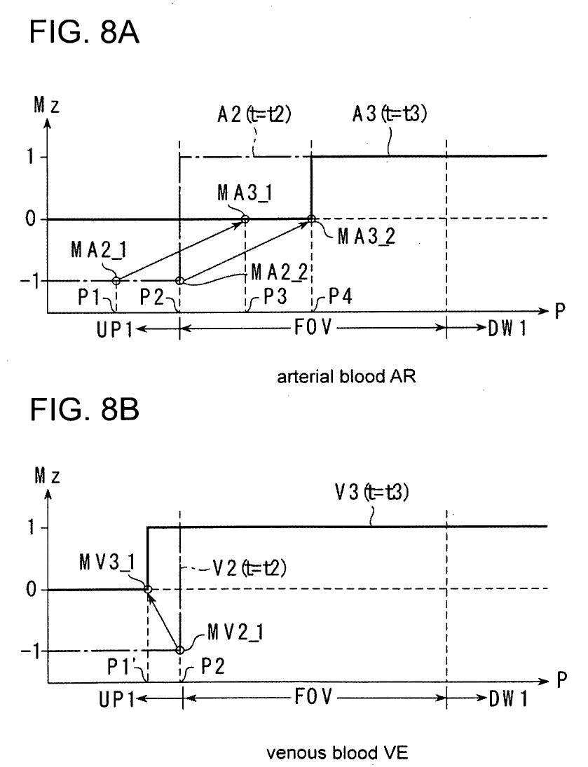

The first inversion period IR1 is followed by the second inversion period IR2 (see FIG. 5). The first inversion time TIa is provided as a time interval between the first inversion period IR1 and the second inversion period IR2. Accordingly, the longitudinal magnetization components Mz of the arterial blood AR and the venous blood VE change during the first inversion time TIa as shown in graphs of FIG. 8.

FIGS. 8A and 8B indicate longitudinal magnetization components Mz of the arterial blood AR and the venous blood VE immediately before the second inversion period IR2 (time t3 in FIG. 5).

The graph in FIG. 8A shows a line A3 (solid line) that represents relation between the position P and the longitudinal magnetization component Mz of the arterial blood AR at time t3. In the graph of FIG. 8A, a dash-dot-line indicates the line A2 in FIG. 7A.

The graph in FIG. 8B shows a line V3 (solid line) that represents relation between the position P and the longitudinal magnetization component Mz of the venous blood VE at time t3. In the graph of FIG. 8B, a dash-dot-line indicates the line V2 in FIG. 7B.

At time t2, the longitudinal magnetization component Mz of the arterial blood AR in the first upstream region UP1 is set to −1 (see line A2). The arterial blood AR, with Mz set to −1, is subject to longitudinal magnetization recovery during the first inversion time TIa. The first embodiment configures inversion time TIa equivalent to a time period (approximately 840 ms) during which the longitudinal magnetization component Mz of the arterial blood AR reaches a null point from −1. The arterial blood AR with Mz set to −1 at time t2 is subject to longitudinal magnetization recovery virtually to the null point immediately before the second inversion period IR2 (time t3). The arterial blood AR flows from the first upstream region UP1 to the imaging region FOV. The longitudinal magnetization component Mz of the arterial blood AR changes from the line A2 (time t2) to the line A3 (time t3). For example, a flow of the arterial blood AR changes a longitudinal magnetization component MA2_1 at P1, a position P on the line A2, to a longitudinal magnetization component MA3_1 at P3, a position P on the line A3. A flow of the arterial blood AR changes a longitudinal magnetization component MA2_2 at P2, a position P on the line A2, to a longitudinal magnetization component MA3_2 at P4, a position P on the line A3.

The arterial blood AR in the second upstream region UP2 (see FIG. 4) flows into the first upstream region UP1, being subject to longitudinal magnetization recovery from Mz=−1 to Mz=0. At time t=t3, the longitudinal magnetization component Mz of the arterial blood AR in the first upstream region UP1 becomes zero.

The venous blood VE with Mz set to −1 at time t2 is also subject to longitudinal magnetization recovery during the first inversion time TIa. A time period for the longitudinal magnetization component Mz of the venous blood VE to reach the null point from −1 is virtually the same as the arterial blood AR. Accordingly, the venous blood VE with Mz set to −1 at time t2 is subject to longitudinal magnetization recovery virtually to the null point immediately before the second inversion period IR2 (time t3). The venous blood VE flows slower than the arterial blood AR in a direction opposite to the arterial blood AR. The longitudinal magnetization component Mz of the venous blood VE changes to the line V3 (time t3) from the line V2 (time t2). For example, a flow of the venous blood VE changes a longitudinal magnetization component MV2_1 at P2, a point P on the line V2, to a longitudinal magnetization component MV3_1 at P1′, a point P on the line V3.

The venous blood VE in the second downstream region DW2 (seed FIG. 4) maintains Mz set to 1 at time t2 and flows into the first downstream region DW1 by keeping Mz=1. Accordingly, the longitudinal magnetization component Mz of the venous blood VE in the first downstream region DW1 is set to 1 at time t3.

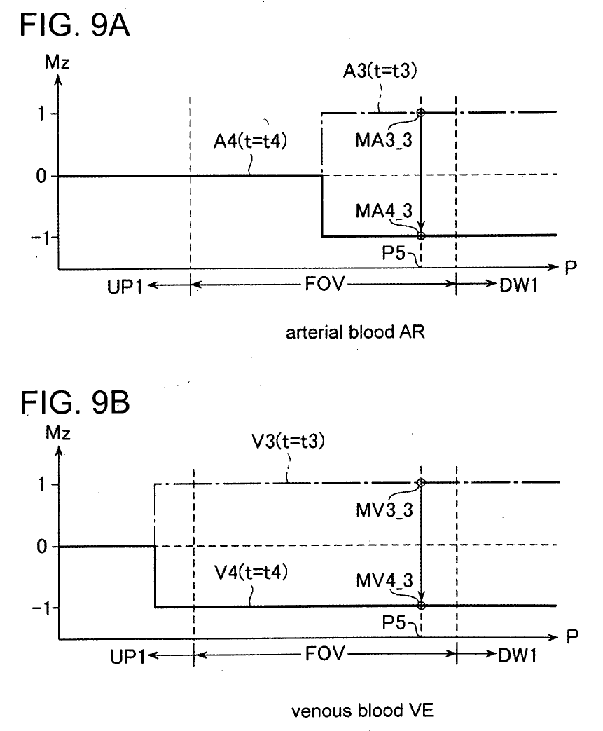

Immediately after time t3, the second inversion period IR2 starts (see FIG. 5). The nonselective RF inversion pulse P2 is applied during the second inversion period IR2. Applying the nonselective RF inversion pulse P2 changes longitudinal magnetization components Mz of the arterial blood AR and the venous blood VE as shown in graphs of FIG. 9.

FIGS. 9A and 9B show longitudinal magnetization component Mz of the arterial blood AR and the venous blood VE immediately after the second inversion period IR2 (time t4 in FIG. 5).

The graph in FIG. 9A shows a line A4 (solid line) that represents relation between the position P and the longitudinal magnetization component Mz of the arterial blood AR at time t4. In the graph of FIG. 9A, a dash-dot-line indicates the line A3 in FIG. 8A.

The graph in FIG. 9B shows a line V4 (solid line) that represents relation between the position P and the longitudinal magnetization component Mz of the venous blood VE at time t4. In the graph of FIG. 9B, a dash-dot-line indicates the line V3 in FIG. 8B.

When the nonselective RF inversion pulse P2 is applied during the second inversion period IR2, the longitudinal magnetization component Mz (longitudinal magnetization direction) of the tissue of the subject 10 is inverted throughout the upstream region UP, the imaging region FOV, and the downstream region DW. As a result, the longitudinal magnetization component Mz of the arterial blood AR is inverted to −1 from 1 and changes to the line A4 from the line A3. Since a time period between times t3 and t4 is sufficiently short, a moving distance of the arterial blood AR from time t3 to time t4 is negligible. For example, a longitudinal magnetization component MA3_3 at P5, a position P on the line A3, changes to a longitudinal magnetization component MA4_3 at P5, a position P on the line A4.

The longitudinal magnetization component Mz (longitudinal magnetization direction) of the venous blood VE also changes to the line V4 from the line V3. Since a time period between times t3 and t4 is sufficiently short, a moving distance of the venous blood VE from time t3 to time t4 is negligible. For example, a longitudinal magnetization component MV3_3 at P5, the position P on the line A3, changes to a longitudinal magnetization component MV4_3 at P5, the position P on the line A4.

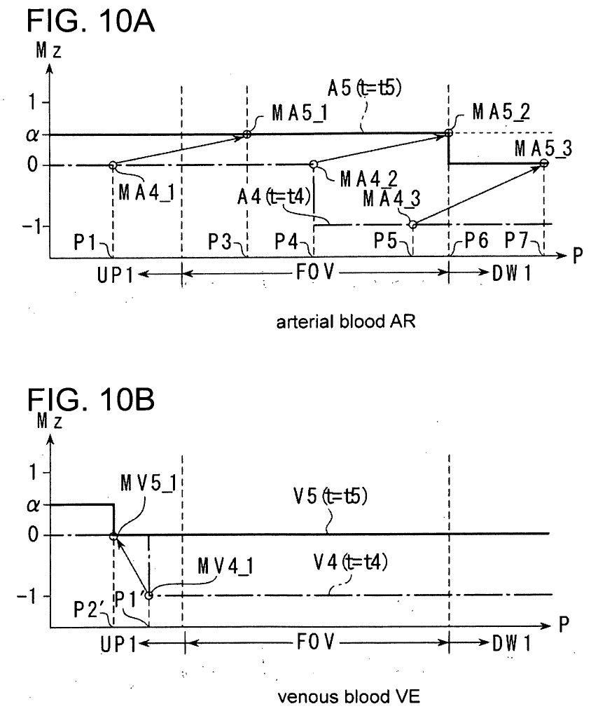

The second inversion period IR2 is followed by the data acquisition period ACQ (see FIG. 5). The second inversion time TIb is provided as a time interval between the second inversion period IR2 and the data acquisition period ACG. Accordingly, the longitudinal magnetization components Mz of the arterial blood AR and the venous blood VE change during the second inversion time TIb as shown in graphs of FIG. 10.

FIGS. 10A and 10B indicate longitudinal magnetization components Mz of the arterial blood AR and the venous blood VE immediately before the data acquisition period ACQ (time t5 in FIG. 5).

The graph in FIG. 10A shows a line A5 (solid line) that represents relation between the position P and the longitudinal magnetization component Mz of the arterial blood AR at time t5. In the graph of FIG. 10A, a dash-dot-line indicates the line A4 in FIG. 9A.

The graph in FIG. 10B shows a line V5 (solid line) that represents relation between the position P and the longitudinal magnetization component Mz of the venous blood VE at time t5. In the graph of FIG. 10B, a dash-dot-line indicates the line V4 in FIG. 10B.

The following describes FIG. 10 in the order of FIG. 10B and then FIG. 10A.

The venous blood VE with Mz set to −1 at time t4 is subject to longitudinal magnetization recovery during the second inversion time TIb. The first embodiment configures the second inversion time TIb equivalent to a time period during which the longitudinal magnetization component Mz of the venous blood VE reaches the null point from −1. In the range of P>P1′ on the line V4, the longitudinal magnetization component Mz of the venous blood VE is subject to longitudinal magnetization recovery from −1 and virtually reaches the null point immediately before the data acquisition period (time t5). The venous blood VE flows in a direction opposite to the arterial blood AR. The longitudinal magnetization component Mz of the venous blood VE changes to the line V5 (time t5) from the line V4 (time t4). For example, a flow of the venous blood VE changes a longitudinal magnetization component MV4_1 at P1′, a point P on the line V4, to a longitudinal magnetization component MV5_1 at P2′, a point P on the line V5. As seen from FIG. 10B, the venous blood VE in the imaging region FOV contains the longitudinal magnetization component Mz set to zero at time t5.

The venous blood VE in the second downstream region DW2 also flows into the first downstream region DW1, being subject to longitudinal magnetization recovery from Mz=−1 to Mz=0 (null point). At time t=t5, the longitudinal magnetization component Mz of the venous blood VE in the downstream region DW1 becomes zero.

The arterial blood AR with Mz set to −1 at time t4 is also subject to longitudinal magnetization recovery during the second inversion time TIb. A time period for the longitudinal magnetization component Mz of the arterial blood AR to reach the null point from −1 is virtually the same as the venous blood VE. In consideration for the blood flow of the arterial blood AR, the longitudinal magnetization recovery changes the longitudinal magnetization component Mz (=1) in the range of P>P4 on the line A4 (time t4) to the longitudinal magnetization component Mz (=0) in the range of P>P6 on the line A5 (time t5). For example, a longitudinal magnetization component MA4_3 at P5, a point P on the line A4, changes to a longitudinal magnetization component MA5_3 at P7, a point P on the line A5.

The longitudinal magnetization component Mz is set to 0 in the range of P<P4 on the line A4. Accordingly, the longitudinal magnetization component Mz becomes greater than 0 during the second inversion time TIb in the range of P<P4 on the line A4 and is subject to longitudinal magnetization recovery up to α (0<α<1). The embodiment defines α as approximately 0.5. In consideration for the blood flow direction of the arterial blood AR, the longitudinal magnetization component Mz (=0) in the range of P<P4 on the line A4 changes to the longitudinal magnetization component Mz (=α=0.5) in the range of P<P6 on the line A5. For example, the longitudinal magnetization component MA4_1 at position P=P1 on the line A4 changes to the longitudinal magnetization component MA5_1 at position P=P3 on the line A5. The longitudinal magnetization component MA4_2 at position P=P4 on the line A4 changes to the longitudinal magnetization component MA5_2 at position P=P6 on the line A5. As seen from FIG. 10A, the arterial blood AR in the imaging region FOV contains the longitudinal magnetization component Mz set to α (=0.5) at time t5.

The arterial blood AR in the second upstream region UP2 also flows into the first upstream region UP1, being subject to longitudinal magnetization recovery from Mz=0 to Mz=α (=0.5). At time t=t5, the longitudinal magnetization component Mz of the arterial blood AR in the upstream region UP1 becomes α (=0.5).

When comparing FIG. 10A with FIG. 10B with respect to the range of the imaging region FOV, the longitudinal magnetization component Mz of the arterial blood AR is set to α (=0.5) on the line A5, however, the longitudinal magnetization component Mz of the venous blood VE is set to zero on the line A5. Accordingly, performing the pulse sequence 50 in FIG. 5 can obtain a blood flow image with the arterial blood AR emphasized.

According to the above-mentioned embodiment, the selective RF inversion pulse P1 is applied before the nonselective RF inversion pulse P2. There is known the Time-SLIP technique that applies a selective RF inversion pulse after a nonselective RF inversion pulse. However, this Time-SLIP technique images the arterial blood AR only in a range narrower than the imaging region FOV obtained in the embodiment. The reason is described below by comparing the embodiment with the Time-SLIP technique.

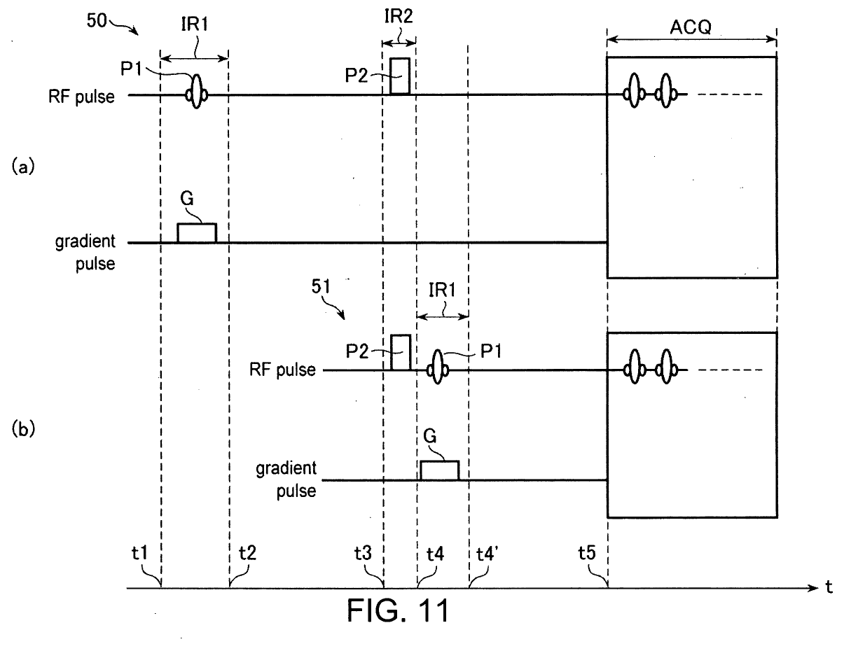

FIGS. 11A and 11B show pulse sequences according to the above-mentioned embodiment and the Time-SLIP technique.

FIG. 11A shows the pulse sequence 50 according to embodiment (see FIG. 5). FIG. 11B shows an example of a pulse sequence according to the Time-SLIP technique.

A pulse sequence 51 according to the Time-SLIP technique is provided with the second inversion period IR2 at the same timing as the pulse sequence 50 according to the embodiment of the invention. However, the first inversion period IR1 is provided immediately after the second inversion period IR2.

The following describes how performing the pulse sequences 50 and 51 changes the longitudinal magnetization component of the arterial blood AR.

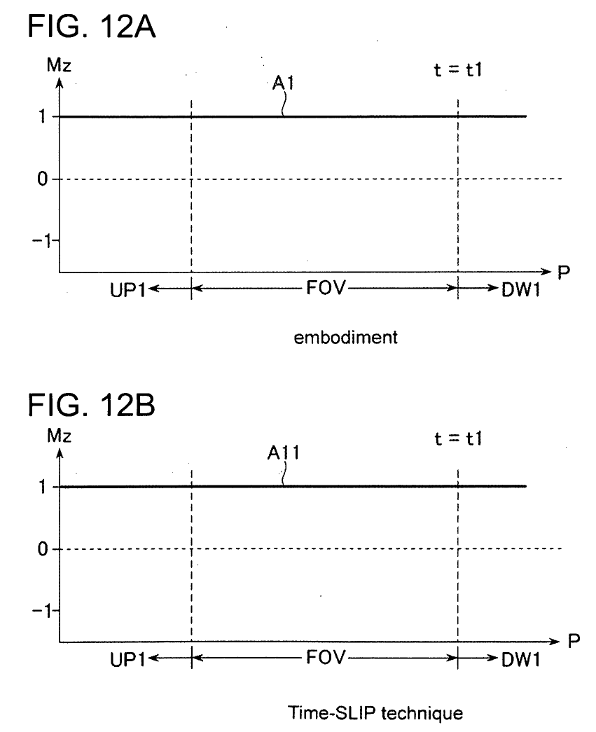

FIGS. 12 through 17 are graphs showing the longitudinal magnetization component Mz of the arterial blood AR of the subject 10 at time points of the pulse sequences 50 and 51 in FIG. 11.

Horizontal axes of the graphs in FIGS. 12 through 17 indicate positions P in the first upstream region UP1, the imaging region FOV, and the first downstream region DW1 (see FIG. 4). Vertical axes of the graphs in FIGS. 12 through 17 indicate longitudinal magnetization components Mz of the arterial blood AR of the subject 10.

FIGS. 12A, 13A, 14A, 15A, 16A, and 17A show longitudinal magnetization components Mz of the arterial blood AR at time points for performing the pulse sequence 50 (see FIG. 11A) according to the embodiment. FIGS. 12B, 13B, 14B, 15B, 16B, and 17B show longitudinal magnetization components Mz of the arterial blood AR at time points for performing the pulse sequence 51 (see FIG. 11B) according to the Time-SLIP technique.

The pulse sequence 51 according to the Time-SLIP technique applies no pulse until time t3. Accordingly, as shown in the FIGS. 12B, 13B, and 14B, in the Time-SLIP technique, the longitudinal magnetization component Mz of the arterial blood AR at times t1, t2, and t3 is 1 throughout the first upstream region UP1, the imaging region FOV, and the first downstream region DW1.

The second inversion period IR2 starts immediately after time t3.

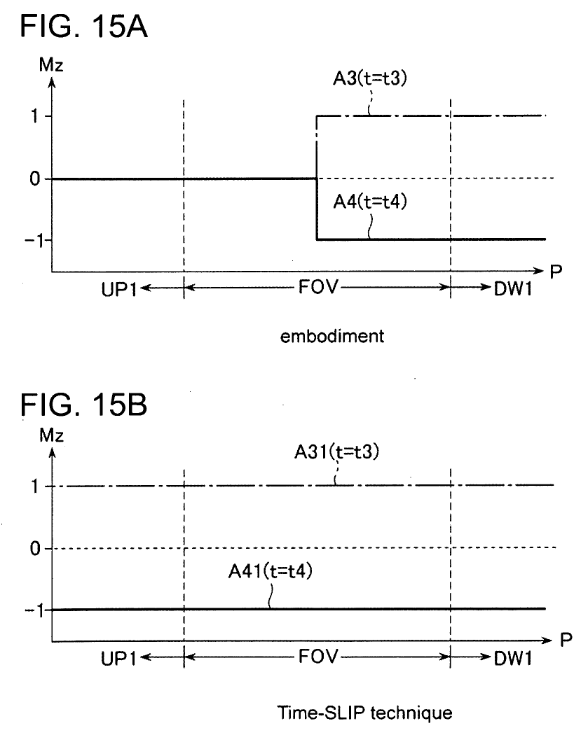

The nonselective RF inversion pulse P2 is applied during the second inversion period IR2. When the nonselective RF inversion pulse P2 is applied, the longitudinal magnetization component Mz of the arterial blood AR changes as shown in the graph of FIG. 15.

FIGS. 15A and 15B show the longitudinal magnetization component Mz of the arterial blood AR immediately after the second inversion period IR2 (time t4 in FIG. 11).

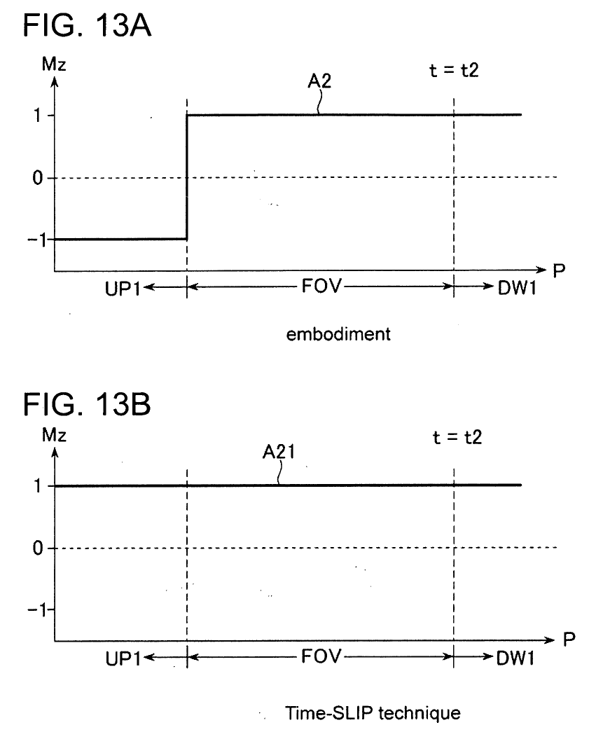

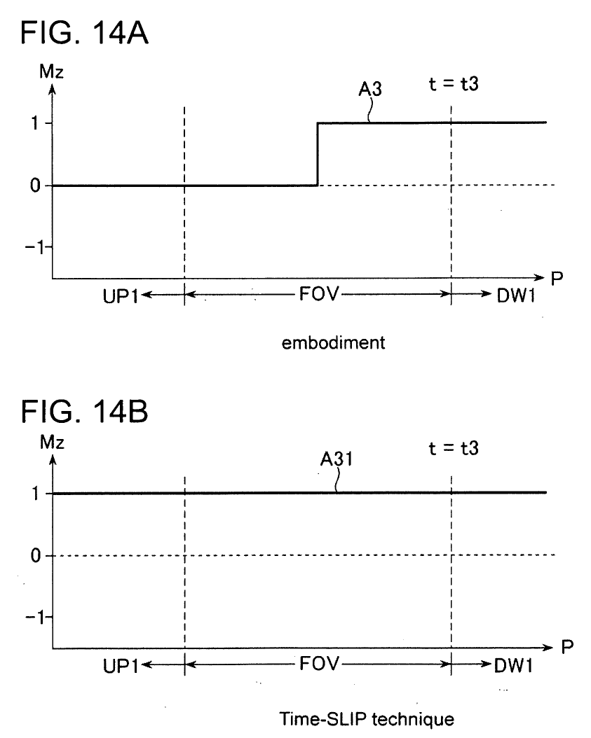

FIG. 15A is the graph according to the embodiment and shows the line A4 (solid line) that represents relation between the position P and the longitudinal magnetization component Mz of the arterial blood AR at time t4. In the graph of FIG. 15A, a dash-dot-line indicates the line A3 in FIG. 14A.

FIG. 15B is the graph according to the Time-SLIP technique and shows a line A41 (solid line) that represents relation between the position P and the longitudinal magnetization component Mz of the arterial blood AR at time t4. In the graph of FIG. 15B, a dash-dot-line indicates a line A31 in FIG. 14B.

When a nonselective RF inversion pulse P2 is applied during the second inversion period IR2, the longitudinal magnetization component Mz (longitudinal magnetization direction) of the tissue of the subject 10 is inverted throughout the upstream region UP, the imaging region FOV, and the downstream region DW. As a result, the longitudinal magnetization component Mz of the arterial blood AR is inverted to −1 from 1. In FIG. 15A (according to the embodiment), the line A3 changes to the line A4. In FIG. 15B (according to the Time-SLIP technique), the line A31 changes to the line A41. When comparing FIGS. 15A with 15B, the longitudinal magnetization component Mz is set to not only −1 but also 0 in the imaging region FOV in FIG. 15A. However, in FIG. 15B, the longitudinal magnetization component Mz is set to −1 throughout the entire imaging region FOV.

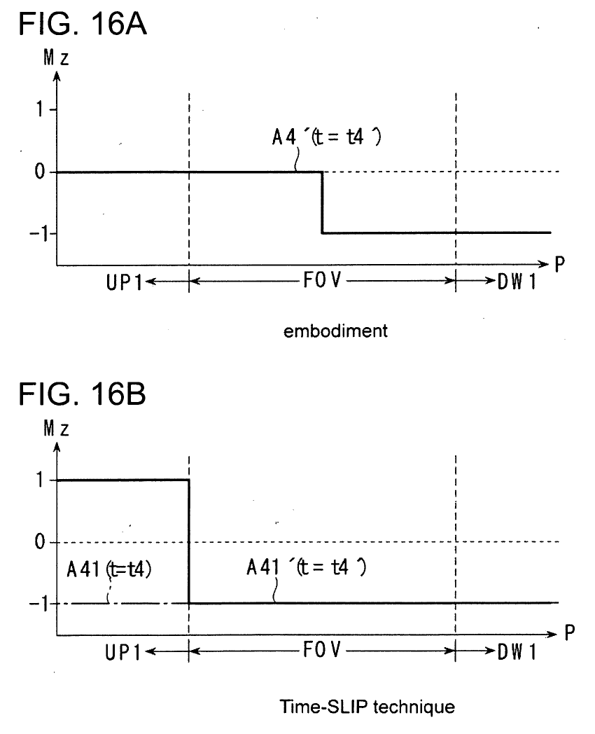

The Time-SLIP technique provides the first inversion period IR1 immediately after the second inversion period IR2. When the selective RF inversion pulse P1 is applied during the first inversion period IR1, the Time-SLIP technique changes the longitudinal magnetization component Mz of the arterial blood AR as shown in the graph of FIG. 16.

FIGS. 16A and 16B show longitudinal magnetization components Mz of the arterial blood AR at time t4′.

FIG. 16A is a graph showing the longitudinal magnetization component Mz of the arterial blood AR at time t4′ according to the embodiment. FIG. 16B is a graph showing the longitudinal magnetization component Mz of the arterial blood AR at time t4′ according to the Time-SLIP technique.

The Time-SLIP technique reverses the longitudinal magnetization direction of a tissue in the upstream region UP during the first inversion period IR1. As shown in FIG. 16B, the longitudinal magnetization component Mz of the arterial blood AR is inverted to 1 from −1 in the upstream region UP (first and second upstream regions UP1 and UP2).

By contrast, the embodiment does not apply the selective RF inversion pulse P1 between times t4 and t4′. According to the embodiment, a graph A4′ at time t4′ is virtually the same as the graph A4 (see FIG. 15A) at time t4. Since a time period between times t4 and t4′ is sufficiently short, a moving distance of the arterial blood AR from time t4 to time t4′ is negligible.

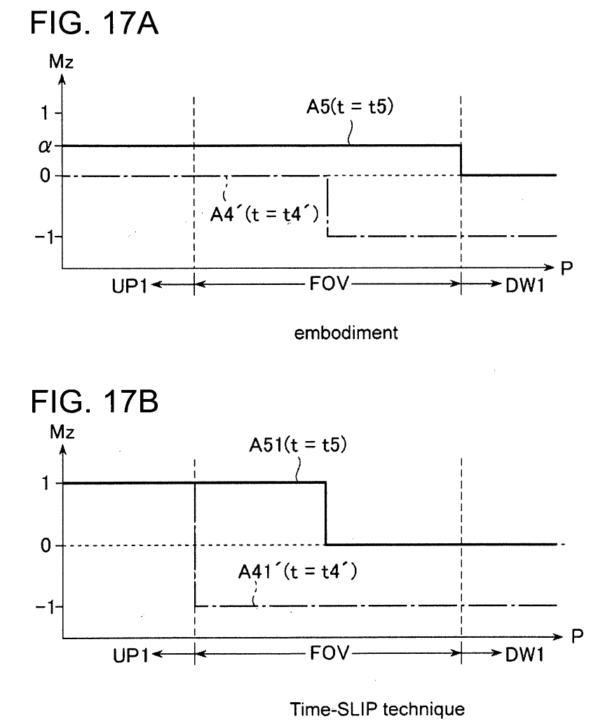

The data acquisition period ACQ starts after time 4′. The second inversion time TIb is provided between the second inversion period IR2 and the data acquisition period ACQ (see FIG. 5). Accordingly, the longitudinal magnetization component Mz of the arterial blood AR changes during the second inversion time TIb as shown in graphs of FIG. 17.

FIGS. 17A and 17B indicate longitudinal magnetization components Mz of the arterial blood AR immediately before the data acquisition period ACQ (time t5 in FIG. 11).

FIG. 17A is a graph showing the longitudinal magnetization component Mz of the arterial blood AR at time t5 according to the embodiment. FIG. 17B is a graph showing the longitudinal magnetization component Mz of the arterial blood AR at time t5 according to the Time-SLIP technique.

FIG. 17A is the graph according to the embodiment and shows the line A5 (solid line) that represents relation between the position P and the longitudinal magnetization component Mz of the arterial blood AR at time t5. In the graph of FIG. 17A, a dash-dot-line indicates the line A4′ in FIG. 16A.

FIG. 17B is the graph according to the Time-SLIP technique and shows a line A51 (solid line) that represents relation between the position P and the longitudinal magnetization component Mz of the arterial blood AR at time t5. In the graph of FIG. 17B, a dash-dot-line indicates a line A41′ in FIG. 16B.

The arterial blood AR with Mz set to −1 at time 4′ recovers to the null point during the second inversion time TIb. In consideration for the blood flow direction of the arterial blood AR, the line A4′ (time t4′) changes to the line A5 (time t5) and the line A41′ (time t4′) changes to the line A51 (time t5).

As shown in FIG. 17B, in the Time-SLIP technique, the longitudinal magnetization component Mz of the arterial blood AR for the left half of the imaging region FOV is 1. However, in the Time-SLIP technique, the longitudinal magnetization component Mz of the arterial blood AR for the right half of the imaging region FOV is 0. Accordingly, it is impossible to visually check the blood flow state of the artery in the right half of the imaging region FOV.

As shown in FIG. 17A, the embodiment provides the longitudinal magnetization component Mz of the arterial blood AR with the value α (=0.5) larger than zero throughout the entire imaging region FOV. Accordingly, the MRI apparatus can image a downstream side of the artery more widely than the Time-SLIP technique.

According to the embodiment (line A5), the longitudinal magnetization component Mz is set to α (=0.5) in the imaging region FOV. Because of α<1, it might be supposed that the arterial blood AR according to the embodiment is less visible than that imaged according to the Time-SLIP technique on the left half of the imaging region FOV. According to the embodiment, however, the longitudinal magnetization component Mz of the arterial blood AR is sized to be α (=0.5). In addition, the longitudinal magnetization component Mz of the venous blood VE is zero (see FIG. 10B). The venous blood VE is actually not rendered in a blood flow image. It is expected that the blood flow state of the artery can be fully visible.

The longitudinal magnetization component Mz of the arterial blood AR is set to α=0.5 when data acquisition starts according to the embodiment (see FIGS. 10A and 17A). A value greater than α can be assigned to the longitudinal magnetization component Mz of the arterial blood AR when data acquisition starts. For example, by shortening the first inversion time TIa, the longitudinal magnetization component Mz of the arterial blood AR at the time of starting data acquisition can be greater than α. The following describes the reason that the longitudinal magnetization component Mz of the arterial blood AR at the time of starting data acquisition can be greater than α by shortening the first inversion time TIa.

Let us consider two inversion times TIa as the first inversion times. One is the inversion time TIa=840 ms similarly to the embodiment. The other inversion time TIa is shorter than 840 ms. The inversion time TIa shorter than 840 ms is assumed to be 600 ms.

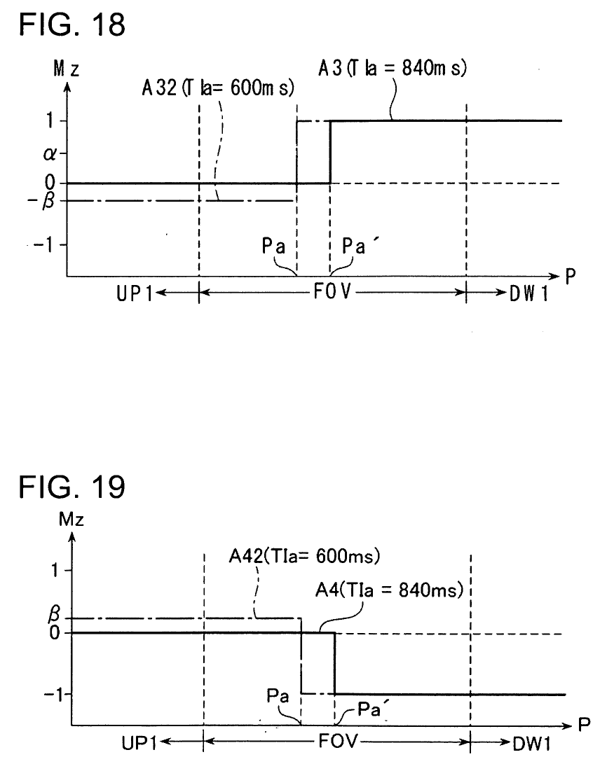

FIGS. 18 through 20 are graphs showing changes in the longitudinal magnetization component Mz of the arterial blood AR at time points of the pulse sequence 50 in FIG. 5 during the two inversion times TIa (840 ms and 600 ms). The longitudinal magnetization component Mz of the arterial blood AR at times t1 and t2 is the same as that in FIGS. 12 and 13 and a description is omitted.

When the inversion times TIa are set to 840 ms and 600 ms, the graph in FIG. 18 shows the longitudinal magnetization component of the arterial blood AR at time t3.

FIG. 18 shows the longitudinal magnetization component Mz of the arterial blood AR at time t3 when the inversion times TIa are set to 840 ms and 600 ms.

The graph in FIG. 18 shows two lines A3 (solid line) and A32 (dash-dot line). The line A3 shows relation between the position P and the longitudinal magnetization component Mz of the arterial blood AR at time t3 when the inversion time TIa is set to 840 ms. The line A32 shows relation between the position P and the longitudinal magnetization component Mz of the arterial blood AR at time t3 when the inversion time TIa is set to 600 ms.

The line A3 with the inversion time TIa set to 840 ms shows that the longitudinal magnetization component Mz reaches the null point in the range of P<Pa′. Since the inversion time TIa is set to 600 ms on the line A32, the longitudinal magnetization component Mz on the line A32 does not reach the null point. On the line A32, Mz is set to −β (0<β<1) in the range of P<Pa.

The second inversion period IR2 is provided immediately after time t3 (see FIG. 5). The nonselective RF inversion pulse P2 is applied during the second inversion period IR2. When the nonselective RF inversion pulse P2 is applied, the longitudinal magnetization component Mz of the arterial blood AR changes as shown in FIG. 19.

FIG. 19 shows the longitudinal magnetization component Mz of the arterial blood AR immediately after the second inversion period IR2 (time t4 in FIG. 5).

The graph in FIG. 19 shows two lines A4 (solid line) and A42 (dash-dot line). The line A4 shows the longitudinal magnetization component Mz of the arterial blood AR at time t4 when the inversion time TIa is set to 840 ms. The line A42 shows the longitudinal magnetization component Mz of the arterial blood AR at time t4 when the inversion time TIa is set to 600 ms.

Applying the nonselective inversion pulse P2 inverts the longitudinal magnetization component Mz (longitudinal magnetization direction) of the arterial blood AR. As a result, the inversion time TIa (=840 ms) causes the longitudinal magnetization component Mz to remain zero in the range of P<P′. The inversion time TIa′ (=600 ms) inverts the longitudinal magnetization component Mz to+β from −β in the range of P<Pa.

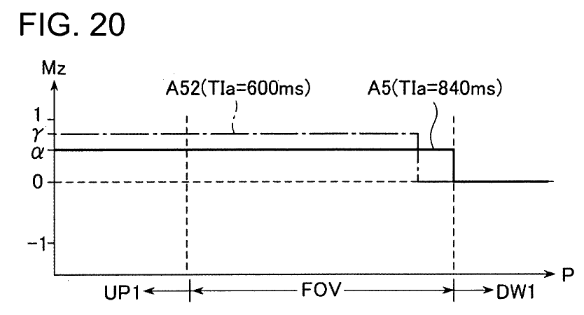

The second inversion period IR2 is followed by the data acquisition period ACQ. The second inversion time TIb is provided as a time interval between the second inversion period IR2 and the data acquisition period ACQ. Similarly to the above-mentioned embodiment, the second inversion time TIb is assumed to be 840 ms. The longitudinal magnetization component Mz of the arterial blood AR changes as shown in a graph of FIG. 20 during the second inversion time TIb.

FIG. 20 shows the longitudinal magnetization component Mz of the arterial blood AR at the start time of the data acquisition period ACQ (time 5 in FIG. 5).

The graph in FIG. 20 shows two lines A5 (solid line) and A42 (dash-dot line). The line A5 shows the longitudinal magnetization component Mz of the arterial blood AR at time t5 when the inversion time TIa is set to 840 ms. The line A42 shows the longitudinal magnetization component Mz of the arterial blood AR at time t5 when the inversion time TIa is set to 600 ms.

The arterial blood AR is subject to longitudinal magnetization recovery during the second inversion period TIb. When the inversion time TIa is set to 840 ms, the longitudinal magnetization component Mz is subject to longitudinal magnetization recovery to α (see FIG. 20) from 0 (see FIG. 19). When the inversion time TIa is set to 600 ms, the longitudinal magnetization component Mz is subject to longitudinal magnetization recovery to γ (see FIG. 20) from β (see FIG. 19). With reference to FIG. 19, the longitudinal magnetization component Mz is set to the null point in the range of P<Pa′ during the inversion time TIa=840 ms. During the inversion time TIa=600 ms, the longitudinal magnetization component Mz is set to β greater than the null point in the range of P<Pa. As shown in FIG. 20, the longitudinal magnetization component Mz for the inversion time TIa=600 ms becomes greater than α and is subject to longitudinal magnetization recovery to γ.

Accordingly, shortening the inversion time TIa can increase the longitudinal magnetization component Mz of the arterial blood AR at the time of starting the data acquisition.

As shown in FIG. 20, however, shortening the inversion time TIa zeros the longitudinal magnetization component Mz of the arterial blood AR in part of the imaging region FOV. Shortening the inversion time TIa narrows the range of the arterial blood AR rendered in the imaging region FOV. It is preferable not to excessively shorten the inversion time TIa when the arterial blood AR needs to be rendered in a wide range. The inversion time TIa can be longer than the time period (840 ms) during which the longitudinal magnetization component Mz of the arterial blood AR reaches the null point from −1.

According to the embodiment, the second inversion time TIb is configured to be equivalent to the time during which the longitudinal magnetization component Mz of the venous blood VE reaches the null point from −1. When the arterial blood AR can be sufficiently separated from the venous blood VE, the second inversion time TIb can be longer or shorter than the time during which the longitudinal magnetization component Mz of the venous blood VE reaches the null point from −1.

To prevent the venous blood VE from being rendered, the embodiment configures the second inversion time TIb to be equivalent to the time during with the longitudinal magnetization component Mz of the venous blood VE reaches the null point from −1. To prevent a tissue (e.g., the kidney 14) other than the venous blood VE from being rendered, the second inversion time TIb just needs to be configured to a time during which the longitudinal magnetization component Mz of the other tissue reaches the null point from −1.

The embodiment renders the arterial blood AR. Further, the invention can render the venous blood VE. To render the venous blood VE, the second inversion time TIb just needs to be configured to a time during which the longitudinal magnetization component Mz of the arterial blood AR or the other tissue (e.g., a motionless tissue) reaches the null point from −1.

According to the embodiment, the nonselective RF inversion pulse P2 is applied during the second inversion period. When the arterial blood AR flowing in the imaging region FOV can be sufficiently rendered, a selective RF inversion pulse may be applied during the second inversion period.

While the embodiment images parts including the kidney 14, the invention can be applied to imaging of the other parts such as a head.

Many widely different embodiments of the invention may be configured without departing from the spirit and the scope of the present invention. It should be understood that the present invention is not limited to the specific embodiments described in the specification, except as defined in the appended claims.

1. An MRI apparatus configured to acquire a blood signal about blood from a subject having the blood flowing from a first region to a second region via an imaging region, said MRI apparatus comprising:
a first longitudinal magnetization inverting device configured to invert a longitudinal magnetization direction of the blood in the first region during a first inversion period;
a second longitudinal magnetization inverting device configured to invert a longitudinal magnetization direction of the blood in process of longitudinal magnetization recovery and to invert a longitudinal magnetization direction of tissues in the imaging region and the second region during a second inversion period after the first inversion period; and
a data acquisition device configured to acquire a blood signal about blood flowing from the first region to the imaging region during a data acquisition period after the second inversion period.

2. The MRI apparatus according to claim 1, wherein:
said first longitudinal magnetization inverting device is configured to apply a first selective RF inversion pulse during the first inversion period; and
said second longitudinal magnetization inverting device is configured to apply one of a nonselective RF inversion pulse a second selective RF inversion pulse during the second inversion period.

3. The MRI apparatus according to claim 2, wherein said second longitudinal magnetization inverting device is configured to apply one of the nonselective RF inversion pulse and the second selective RF inversion pulse after the first selective RF inversion pulse is applied and a first inversion time expires.

4. The MRI apparatus according to claim 3, wherein said data acquisition device is configured to apply an excitation pulse after one of the nonselective RF inversion pulse and the second selective RF inversion pulse is applied and after a second inversion time expires.

5. The MRI apparatus according to claim 2, wherein the first inversion time is a time period from a time after said first longitudinal magnetization inverting device inverts a longitudinal magnetization direction of the blood to a time when a longitudinal magnetization component of the blood reaches a null point.

6. The MRI apparatus according to claim 2, wherein the first inversion time is shorter than a time period from a time after said first longitudinal magnetization inverting device inverts a longitudinal magnetization direction of the blood to a time when a longitudinal magnetization component of the blood reaches a null point.

7. The MRI apparatus according to claim 2, wherein the first inversion time is longer than a time period from a time after said first longitudinal magnetization inverting device inverts a longitudinal magnetization direction of the blood to a time when a longitudinal magnetization component of the blood reaches a null point.

8. The MRI apparatus according to claim 5, wherein the second inversion time is a time period from a time after said second longitudinal magnetization inverting device inverts a longitudinal magnetization direction of a tissue in the imaging region and the second region to a time when a longitudinal magnetization component of the tissue reaches a null point.

9. The MRI apparatus according to claim 6, wherein the second inversion time is a time period from a time after said second longitudinal magnetization inverting device inverts a longitudinal magnetization direction of a tissue in the imaging region and the second region to a time when a longitudinal magnetization component of the tissue reaches a null point.

10. The MRI apparatus according to claim 7, wherein the second inversion time is a time period from a time after said second longitudinal magnetization inverting device inverts a longitudinal magnetization direction of a tissue in the imaging region and the second region to a time when a longitudinal magnetization component of the tissue reaches a null point.

11. The MRI apparatus according to claim 5, wherein the second inversion time is shorter than a time period from a time after said second longitudinal magnetization inverting device inverts a longitudinal magnetization direction of a tissue in the imaging region and the second region to a time when a longitudinal magnetization component of the tissue reaches a null point.

12. The MRI apparatus according to claim 6, wherein the second inversion time is shorter than a time period from a time after said second longitudinal magnetization inverting device inverts a longitudinal magnetization direction of a tissue in the imaging region and the second region to a time when a longitudinal magnetization component of the tissue reaches a null point.

13. The MRI apparatus according to claim 7, wherein the second inversion time is shorter than a time period from a time after said second longitudinal magnetization inverting device inverts a longitudinal magnetization direction of a tissue in the imaging region and the second region to a time when a longitudinal magnetization component of the tissue reaches a null point.

14. The MRI apparatus according to claim 5, wherein the second inversion time is longer than a time period from a time after said second longitudinal magnetization inverting device inverts a longitudinal magnetization direction of a tissue in the imaging region and the second region to a time when a longitudinal magnetization component of the tissue reaches a null point.

15. The MRI apparatus according to claim 6, wherein the second inversion time is longer than a time period from a time after said second longitudinal magnetization inverting device inverts a longitudinal magnetization direction of a tissue in the imaging region and the second region to a time when a longitudinal magnetization component of the tissue reaches a null point.

16. The MRI apparatus according to claim 1, wherein the blood is an arterial blood and the tissue is a venous blood.

17. The MRI apparatus according to claim 1, wherein the blood is an arterial blood and the tissue is a motionless tissue.

18. The MRI apparatus according to claim 1, wherein the blood is a venous blood and the tissue is an arterial blood.

19. The MRI apparatus according to claim 1, wherein the blood is a venous blood and the tissue is a motionless tissue.

20. A magnetic resonance imaging method for acquiring a blood signal about blood from a subject having the blood flowing from a first region to a second region via an imaging region, said method comprising:
inverting a longitudinal magnetization direction of the blood in the first region during a first inversion period;
inverting a longitudinal magnetization direction of the blood in process of longitudinal magnetization recovery;
inverting a longitudinal magnetization direction of tissues in the imaging region and the second region during a second inversion period after the first inversion period; and
acquiring a blood signal about blood flowing from the first region to the imaging region during a data acquisition period after the second inversion period.
